(19) United States
(12) Patent Application Publication
(10) Pub. No.: US 2011/0137050 A1
(43) Pub. Date: Jun. 9, 2011

(54) Process for the Preparation of 4,7-Dimethoxy-5-Methyl-1,3-Benzodioxole and Derivatives Thereof

(75) Inventors: Kai-Yih Tarng, Xin Dian City (TW); Jenn-Tsang Hwang, Hsinchu City (TW)

(73) Assignee: YONG CHUN PROSPEROUS BIOTECH CO., Xin Dian City (TW)

(21) Appl. No.: 12/630,949

(22) Filed: Dec. 4, 2009

(51) Int. Cl. C07D 317/46

(52) U.S. Cl. 549/437

(57) ABSTRACT

Disclosed herein is a novel process for the preparation of a compound of formula (I),

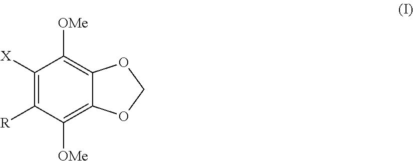

In which a compound of formula (II) is reacted with a Lewis acid to produce a compound of formula (III); and the compound of formula (III) is subsequently reacted with dihalomethane to give the desired compound of formula (I);

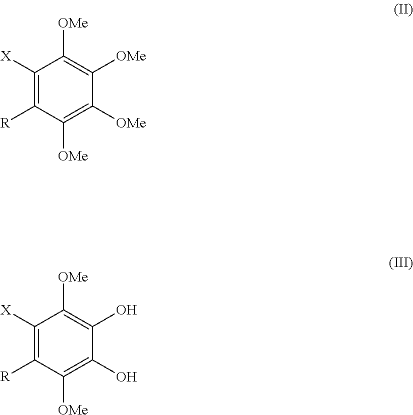

wherein R is nil or C₁₋₆ alkyl, and X is nil, a nitro group, an amino group or a halogen selected from the group consisting of F, Cl, Br and I.

### TECHNICAL FIELD

This disclosure in general relates to a new process for the preparation of 4,7-dimethoxy-5-methyl-1,3-benzodioxole (DMB) and/or its derivatives, which are known for their therapeutic effects in inhibiting the growth of tumor cells.

### BACKGROUND ART

### Description of Related Art

US 2008/0103195 A1 (Liu et al) described compounds isolated from organic solvent extract of Antrodia camphorata have therapeutic effects on human cancers, such as breast cancers, liver cancers and prostate cancers. The identified compounds have a general formula of C₁₀O₄H₁₂, and further comprise several structural formulas, including formula (2) to formula (7), respectively represent 4,7-dimethoxy-5-methyl-1,3-benzodioxole (formula (2)) and its position isomers (formula (3) to (7)).

According to US 2008/0130195 A1, Antrodia camphorata only grows on the inner heartwood wall of the endemic evergreen Cinnamomun Kanehirai (Hay) from an altitude of 450 m to 2000 m in Taiwan and has become rare and difficult to obtain due to illegal logging and the extremely slow growth rate of Antrodia camphorata.

Therefore, there exist in the related art a need to provide therapeutic compounds of 4,7-dimethoxy-5-methyl-1,3-benzodioxole (DMB) and derivatives thereof in a relatively fast and easy manner without using the rare and expensive material, Antrodia camphorata. This invention address such need by providing a process of synthesizing afore-mentioned therapeutic compounds in a 2-steps reaction, which is simple and easy to use, and may be scaled-up to meet industrial applications.

### SUMMARY

As embodied and broadly described herein, disclosure herein features a novel process of producing 4,7-dimethoxy-5-methyl-1,3-benzodioxole (DMB) and/or its derivatives, which are known for their therapeutic efficacy in inhibiting the growth of tumor cells.

Therefore, it is the objective of this disclosure to provide a process of producing a compound of formula (I),

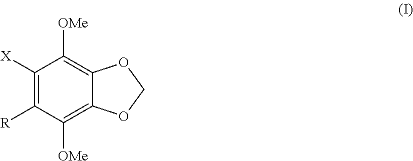

wherein R is nil or C₁₋₆ alkyl, and X is nil, a nitro group, an amino group or a halogen selected from the group consisting of F, Cl, Br and I; the process comprises steps of:

(a) reacting a compound of formula (II) with a Lewis acid to produce a compound of formula (III); and

(b) reacting the compound of formula (III) with dihalomethane to produce the compound of formula (I);

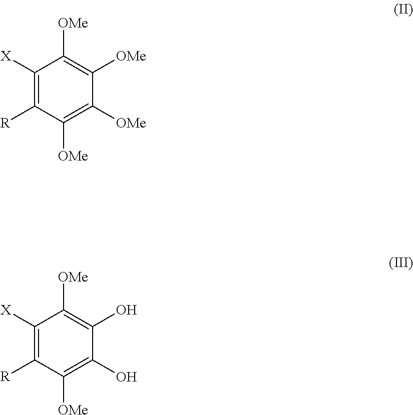

wherein R and X are as defined above.

In step (a), the Lewis acid may be any of AlCl₃, BCl₃, BBr₃, BI₃, NbCl₅, FeCl₃ or lanthanide triflates. In one example, the Lewis acid is AlCl₃; in another example, the Lewis acid is BCl₃.

In step (b), the dihalomethane may be any of dichloromethane (CH₂Cl₂), dibromomethane (CH₂Br₂), diiodomethane (CH₂I₂), bromochloromethane (CH₂BrCl), or bromoiodomethane (CH₂BrI).

According to one specific example, the compound of formula (I) is 4,7-dimethoxy-5-methyl-1,3-benzodioxole (DMB).

In one example, the step (a) is performed under an inner gas such as nitrogen or argon at about 40° C. for at least 16 hours. In another example, the step (b) further comprises adding a base such as cesium carbonate, cesium bicarbonate, cesium fluoride, potassium carbonate, or sodium carbonate, and allows the step (b) to be performed under nitrogen at about 110° C. for at least 16 hours. In one example, the base is cesium carbonate.

The details of one or more embodiments of the invention are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### DISCLOSURE OF INVENTION

The practices of this invention are hereinafter described in detail with respect to a novel process for producing a compound having formula (I),

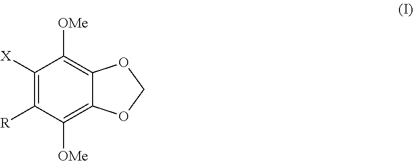

wherein R is nil or C₁₋₆ alkyl, and X is nil, a nitro group, an amino group or a halogen selected from the group consisting of F, Cl, Br and I. The process comprising steps of:

(a) reacting a compound of formula (II) with a Lewis acid to produce a compound of formula (III); and

(b) reacting the compound of formula (III) with dihalomethane to produce the compound of formula (I);

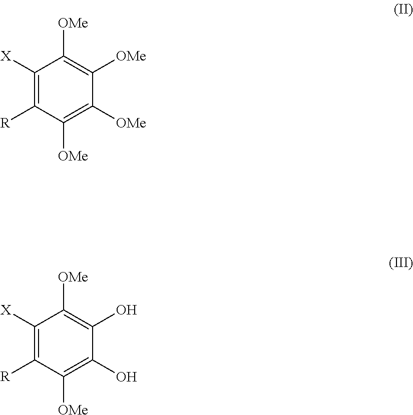

wherein R and X are as defined above.

In step (a), a compound of formula (II) is reacted with a Lewis acid to produce a compound of formula (III), which is used as an intermediate in subsequent step for the preparation of the compound of formula (I). Reaction in step (a) may preferably be carried out at a temperature from about 5° C. to 50° C., such as about 5° C., 10° C., 15° C., 20° C., 30° C., 40° C. or 50° C., for at least 16 hours. A Lewis acid is a compound that may accept a pair of electrons and form a coordinate covalent bond, and therefore is also termed an electrophile or an electron acceptor. Suitable Lewis acid includes, but is not limited to, aluminium chloride, iron (III) chloride, boron trifluoride, boron trichloride and or it's alkylsulfide complex, boron tribromide and or it's alkylsulfide complex, boron triiodide, niobium pentachloride and lanthanide triflates such as ytterbium (III) triflate. In one example, the Lewis acid is AlCl₃; in another example, the Lewis acid is BCl₃. In a preferred example, step (a) is performed at a temperature at about 40° C. for at least 16 hours.

Step (b) may be carried out directly right after step (a). In step (b), the compound of formula (III) obtained from step (a) reaction is further reacted with dihalomethane under nitrogen to produce the desired compound of formula (I). Suitable dihalomethane may include, and is not limited to, dichloromethane (CH₂Cl₂), dibromomethane (CH₂Br₂), diiodomethane (CH₂I₂), bromochloromethane (CH₂BrCl), and bromoiodomethane (CH₂BrI). In one example, step (b) further comprises adding a base such as cesium carbonate, potassium carbonate, or sodium carbonate, and allows the reaction mixture to be performed under nitrogen at a temperature from about 80° C. to about 150° C., such as about 80° C., 90° C., 100° C., 110° C., 120° C., 130, 140° C. or 150° C., for at least 16 hours. The amount of the base may be adjusted empirically according to the reaction scheme, for example, one molecule of DMB requires 2 molecules of the base. Typically, the amount of the added base is usually about 0.5 to 2 moles.

Both reaction steps (a) and (b) are carried out under nitrogen in an inert solvent. Suitable solvents for step (a) are aliphatic or aromatic hydrocarbons, mono- or di-haloalkane, ethers, lactams, sulfoxides, sulfones, nitriles or mixtures thereof. Examples include, but are not limited to, chloromethane, dichloromethane, bromoiodomethane, hexane, cyclohexane, benzene, toluene, xylene, dimethyl ether, diethyl ether, tetrahydrofuran, dioxane, dimethyl sulfoxide, tetramethylenesulfone, acetonitrile and etc. In one example, the solvent used in step (a) is dichloromethane. Solvent that may at the same time be a reactant in step (b), such as, for example, bromoiodomethane (CH₂BrCl), is not added until step (b) and may be added in the form of an excess of other reactants or in addition to other solvent. For example, in step (b), bromochloromethane may be used in excess amount of another reactant, so as to act as a solvent and a reactant at the same time. Alternatively, in step (b), bromochloromethane may be used as a reactant in the presence of a high polar aprotic solvent, including, but is not limited to, dimethyl sulfoxide, N,N-dimethylformamide, N,N-dimethylacetamide, N-methyl-2-pyrrolidinone.

In one preferred process, the compound of formula (I), particularly, 4,7-dimethoxy-5-methyl-1,3-benzodioxole (DMB) is prepared. DMB and its derivatives may be used as pharmaceuticals to inhibit the growth of tumor cells, such as breast cancers, liver cancers and prostate cancers.

It must be noted that as used herein and in the appended claims, the singular forms “a”, “an”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice of the present invention, exemplary methods and materials are described for illustrative purposes.

The following Examples are provided to illustrate certain aspects of the present invention and to aid those of skilled in the art in practicing this invention. These Examples are in no way to be considered to limit the scope of the invention in any manner.

### EXAMPLES

In general, 4,7-dimethoxy-5-methyl-1,3-benzodioxole, was synthesized in accordance with the following scheme via a 2-steps reaction as depicted bellow.

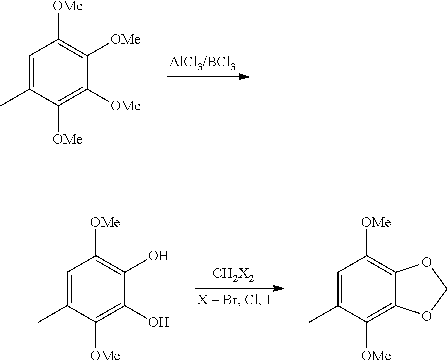

### Example 1

### Preparation of 4,7-dimethoxy-5-methyl-1,3-benzodioxole

### 1.1 Preparation of 3,4-dihydroxy-2,5-dimethoxytoluene

In a flask under nitrogen, 0.94 g of 2,3,4,5-tetramethoxytoluene (4.43 mmole) were dissolved in anhydrous dichloromethane (12 ml). 1.30 g of AlCl₃ (9.77 mmole) were added in 3 portions, with a 5-minute interval between each portion, under nitrogen and the resulted mixture was stirred at room temperature for 30 minutes. Then, the mixture was heated to 40° C. and reacted for at least 16 hours. Allowed the temperature to return to the room temperature, then poured the mixture into 60 ml of iced water, and extracted with dichloromethane for at least 3 times, each time using 20 ml of dichloromethane. The organic layer was washed with saturated NaCl solution, dried over Na₂SO₄, filtered to remove residual solvent, and 2.6 g of a brown oily raw product was obtained. The raw product was further purified by column chromatography (the column was eluted with ethyl acetate/hexane=1/4 to 1/1) to result a pure product of 3,4-dihydroxy-2,5-dimethoxytoluene (0.91 g, yield: 31%).

TLC R_{f}: 0.28 (EtOAc/hexane 1:1)

¹HNMR (500 MHz, CDCl₃) δ 2.22 (s, 3H), 3.78 (s, 3H), 3.82 (s, 3H), 5.48 (br s, 1H), 5.66 (br s, 1H), 6.24 (s, 1H); and

¹³C NMR (125 MHz, CDCl₃) δ 15.47, 56.23, 60.63, 103.99, 120.92, 131.54, 137.05, 140.30, 143.39.

### 1.2 Preparation of 4,7-dimethoxy-5-methyl-1,3-benzodioxole

240 mg of the compound of example 1.1 (1.35 mmole) was dissolved in 3 ml of dimethyl sulfoxide (DMSO), 227 mg of bromochloromethane (1.76 mmol) and 442 mg of Cs₂CO₃ (1.35 mmole) were then added. The mixture was then heated to 110° C. in an oil bath under nitrogen, and allowed the reaction to proceed for 16 hours. Let the mixture cool down then diluted the mixture by adding 15 ml of water. Extracted the mixture with isopropanol (5 ml/each time, for 3 times), washed the extract with saturated NaCl solution, then dried over Na₂SO₄, filtered to remove residual solvent, and 250 mg of a dark brown raw product was obtained. The raw product was further purified by column chromatography (the column was eluted with ethyl acetate/hexane=1/5) to result a colorless liquid of 4,7-dimethoxy-5-methylbenzo[1,3]dioxole (85 mg, yield: 69.8%).

TLC: R_{f}=0.52 (EtOAc/hexane 1:2)

¹HNMR (500 MHz, CDCl₃) δ 2.17 (s, 3H), 3.84 (s, 3H), 3.88 (s, 3H), 5.93 (s, 1H), 6.30 (s, 1H); and

¹³C NMR (125 MHz, CDCl₃) δ 15.78, 56.80, 59.78, 101.32, 108.82, 123.54, 134.61, 136.47, 138.55, 138.76; MS (m/e): 197 (M+H).

### Example 2

### Preparation of 4,7-dimethoxy-5-methyl-1,3-benzodioxole

### 2.1 Preparation of 3,4-dihydroxy-2,5-dimethoxytoluene

3.36 g of the compound of example 1.1 (15.85 mmole) was dissolved in 40 ml of anhydrous dichloromethane; and 35 ml of BCl₃ (1M in hexane, 35 mmole) were then added over a period of 30 minutes, and the mixture was stirred under nitrogen in an iced bath for 2 hours. Then, the mixture was warmed up to room temperature and allowed the reaction to proceed for 24 hours. The mixture was then poured into 150 ml of iced water, filtered to remove solvent, and 2.6 g of brown oily raw product was obtained. The column was eluted with proper solutions (ethyl acetate/hexane is between 1/4 to 1/1) and a pure product of 3,4-dihydroxy-2,5-dimethoxytoluene was obtained (0.91 g; yield: 31%).

TLC R_{f}: 0.28 (EtOAc/hexane 1:1)

¹HNMR (500 MHz, CDCl₃) δ 2.22 (s, 3H), 3.78 (s, 3H), 3.82 (s, 3H), 5.48 (br s, 1H), 5.66 (br s, 1H), 6.24 (s, 1H); and

¹³C NMR (125 MHz, CDCL₃) δ 15.47, 56.23, 60.63, 103.99, 120.92, 131.54, 137.05, 140.30, 143.39.

### 2.2 Preparation of 4,7-dimethoxy-5-methyl-1,3-benzodioxole

4,7-dimethoxy-5-methylbenzo[1,3]dioxole was synthesized in accordance with the steps described in Example 1.2 using 3,4-dihydroxy-2,5-dimethoxytoluene of Example 2.1, and a pure product of 3,4-dihydroxy-2,5-dimethoxytoluene was obtained (85 mg, yield: 69.8%).

TLC: R_{f}=0.52 (EtOAc/hexane 1:2)

¹HNMR (500 MHz, CDCl₃) δ 2.17 (s, 3H), 3.84 (s, 3H), 3.88 (s, 3H), 5.93 (s, 1H), 6.30 (s, 1H); and

¹³C NMR (125 MHz, CDCl₃) δ 15.78, 56.80, 59.78, 101.32, 108.82, 123.54, 134.61, 136.47, 138.55, 138.76; MS (m/e): 197 (M+H).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A process of producing a compound of formula (I), 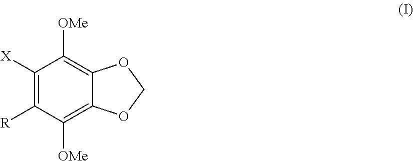
wherein R is H or C₁₋₆ alkyl, and X is H, a nitro group, an amino group or a halogen selected from the group consisting of F, Cl, Br and I; the method comprising:
(a) reacting a compound of formula (II) with a Lewis acid to produce a compound of formula (III); and
(b) reacting the compound of formula (III) with dihalomethane to produce the compound of formula (I); 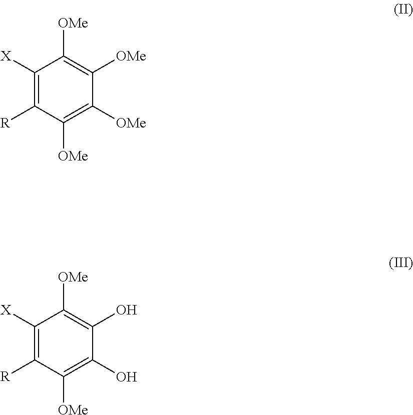
wherein R and X are as defined above.

2. The process of claim 1, wherein the Lewis acid is any of AlCl₃, BCl₃, BBr₃, BI₃, NbCl₅, FeCl₃ or lanthanide triflates.

3. The process of claim 1, wherein the dihalomethane is any of dichloromethane (CH₂Cl₂), dibromomethane (CH₂Br₂), diiodomethane (CH₂I₂), bromochloromethane (CH₂BrCl), or bromoiodomethane (CH₂BrI).

4. The process of claim 1, wherein R is methyl and X is H.

5. The process of claim 1, wherein the step (a) is performed under nitrogen at about 40° C. for at least 16 hours.

6. The process of claim 1, wherein the step (b) further comprises adding a base and allows the step (b) to proceed under nitrogen in an inner solvent at about 110° C. for at least 16 hours.

7. The process of claim 6, wherein the base is any of cesium carbonate (Cs₂CO₃), cesium bicarbonate (CsHCO₃), cesium fluoride (CsF), potassium carbonate (K₂CO₃) or sodium carbonate (Na₂CO₃).

8. The process of claim 1, wherein step (a) is performed in an inner solvent that is selected from the group consisting of chloromethane, dichloromethane, bromoiodomethane, hexane, cyclohexane, benzene, toluene, xylene, dimethyl ether, diethyl ether, tetrahydrofuran, dioxane, tetramethylenesulfone, acetonitrile and dimethyl sulfoxide.

9. The process of claim 1, wherein step (b) is performed in an inner solvent that is selected from the group consisting of dimethyl sulfoxide, N,N-dimethylformamide, N,N-dimethylacetamide and N-methyl-2-pyrrolidinone.
